(19) United States
(12) Patent Application Publication
(10) Pub. No.: US 2014/0261416 A1
(43) Pub. Date: Sep. 18, 2014

(54) THERMAL COOLER & DEHUMIDIFIER FOR EXHALATION PATH IN VENTILATOR SYSTEM

(71) Applicant: KONINKLIJKE PHILIPS N.V., EINDHOVEN (NL)

(72) Inventors: Mabini Arcilla, San Diego, CA (US); Smita Garde, Irvine, CA (US); Samir Ahmad, San Diego, CA (US)

(21) Appl. No.: 14/353,788

(22) PCT Filed: Oct. 26, 2012
(86) PCT No.: PCT/IB2012/055906

§ 371 Date: Apr. 24, 2014

Related U.S. Application Data
(60) Provisional application No. 61/552,878, filed on Oct. 28, 2011.

(51) Int. Cl. A61M 16/10; A61M 16/08; A61M 16/00; A61M 16/16

(52) U.S. Cl. CPC A61M 16/1085; A61M 16/16; A61M 16/107; A61M 16/1065; A61M 16/0891; A61M 16/0051; A61M 16/0057
USPC 128/203.14; 128/203.12

(57) ABSTRACT

A ventilator system (110) includes: an inhalation filter (120) configured to receive and filter a gas; a humidifier (130) connected to the inhalation filter and configured to adjust a humidity of the filtered gas; a dual-limb patient circuit connected to a patient and configured to supply ventilation to the patient, the dual limb patient circuit including an inspiratory limb connected to the humidifier and configured to supply the filtered gas to the patient and an expiratory limb configured to receive an exhaled gas from the patient; a condenser (140) connected to the expiratory limb, the condenser being configured to cool and remove moisture from the gas from the patient; and an expiratory filter (150) connected to the condenser and configured to filter the cooled gas from the condenser.

### TECHNICAL FIELD

This invention pertains to patient ventilator systems and apparatus, and in particular, a thermal cooler and dehumidifier for the exhalation path of a ventilator system, and a ventilation system that includes such a thermal cooler and dehumidifier.

### BACKGROUND AND SUMMARY

Ventilators are used in a variety of settings. For example, in a hospital a patient may be ventilated as part of their medical care. In particular, ventilators are commonly provided in hospital intensive care units (ICUs).

Expiratory filters are filters that are used to filter exhaled gas from a patient in the expiratory limb of a dual-limb patient circuit used with a ventilator. The purpose of the filter is to reduce cross-contamination of the ventilator between patients. For example, the bacterial and viral filtration efficiency of such an expiratory filter is rated according to NIOSH (e.g., N100 filters remove 99.97% of particles in an exhaled gas).

In general, expiratory filters used with a ventilator can be divided into heated expiratory filters and non-heated expiratory filters.

When a non-heated expiratory filter is employed, humidity from the exhaled gas and/or from active humidification causes the filter media to absorb moisture. As the filter media accumulates moisture, its resistance to gas flow increases, which may lead to increased work for the patient to breathe. As the non-heated expiratory filter accumulates moisture, it becomes wet and contaminated and needs to be replaced—typically after every 24 hours of use. This causes a break in the patient circuit during replacement of the expiratory filter. Also, flow sensors in some ventilators that are employed for measuring the exhaled flow of gas may be susceptible to damage when exhaled gas passing through the sensor has a high humidity level, which may be the case when a non-heated expiratory filter is employed.

On the other hand, a heated expiratory filter keeps the filter media dry and does not need to be replaced, and thus it can avoid a break in the patient circuit. In addition to keeping the filter media dry, a heated filter helps in the measurement of the exhaled flow of gas when exhaled gas passing through the sensor has a high humidity level. However, in general a heated expiratory filter requires control input(s) from a ventilator, and is typically integrated with the ventilator. So, in general, heated expiratory filters may only be employed when a ventilator includes a heated ventilation filter control. Furthermore, I general a heated expiratory filter may substantially increase the power consumption of a ventilator system.

Accordingly, it would be desirable to provide a device which can address one or more of the issues described above.

In one aspect of the invention, a system comprises: an inhalation filter configured to receive and filter a gas; a humidifier connected to the inhalation filter and configured to adjust a humidity of the filtered gas; a dual-limb patient circuit connected to a patient and configured to supply ventilation to the patient, the dual limb patient circuit including an inspiratory limb connected to the humidifier and configured to supply the filtered gas to the patient, and further including an expiratory limb configured to receive an exhaled gas from the patient; a condenser connected to the expiratory limb, the condenser being configured to cool and remove moisture from the exhaled gas from the patient; and an expiratory filter connected to the condenser and configured to filter the cooled exhaled gas from the condenser.

In some embodiments, the condenser comprises: an inlet configured to receive the exhaled gas from the patient; an outlet configured to output the cooled exhaled gas from the condenser; a reservoir connected to the inlet and to the outlet; and a heat remover configured to cool the exhaled gas within the reservoir.

In some embodiments, the heat remover is a thermoelectric cooling device.

In some embodiments, the system further comprises a thermally conductive cold plate thermally coupled to the thermoelectric cooling device and substantially surrounding the reservoir.

In some embodiments, the reservoir comprises: a canister disposed within a cavity formed by the thermally conductive cold plate; a disposable liner removably disposed within the canister; and an O-Ring coupling the disposable liner to the canister.

In some embodiments, the system further comprises a spiral fin disposed within the disposable liner such that at least a portion of the exhaled gas from the patient passes along the spiral fin in a path from the inlet to the outlet.

In some embodiments, the system further comprises a gas flow unit comprising the inlet, the outlet, the spiral fin, and an exhaust tube connected to the inlet and disposed within the disposable liner through which the exhaled gas from the patient passes into the disposable liner, and wherein the spiral fin is provided on an external surface of the exhaust tube.

In some embodiments, the condenser includes a control input configured to receive a control signal from a ventilator for controlling a cooling operation of the thermoelectric cooling device.

In some embodiments, the condenser includes a manual control for controlling a cooling operation of the thermoelectric cooling device, and wherein the manual control is configured to be manually adjusted by a user.

In some embodiments, the system further comprises a ventilator connected to the inhalation filter and to the expiratory filter, and configured to provide the gas to the inhalation filter and to receive the filtered exhaled gas from the expiratory filter.

In another aspect of the invention, an apparatus comprises: an inlet configured to be connected to an expiratory limb of a dual-limb patient circuit; an outlet configured to be connected to an expiratory filter of a ventilator system; a thermoelectric cooler; a thermally conductive cold plate thermally coupled to the thermoelectric cooling device and having a cavity formed therein; a reservoir disposed within the cavity and coupled to the inlet and the outlet; and a spiral fin disposed within the reservoir in a gas flow path from the inlet to the outlet.

In some embodiments, the canister includes a monitor window through which an interior of the canister may be viewed from outside the canister when the canister is disposed within the cavity.

In yet another aspect of the invention, an apparatus comprises: an inlet configured to be connected to an expiratory limb of a dual-limb patient circuit and configured to receive a gas therefrom; an outlet configured to be connected to an expiratory filter of a ventilator system and configured to provide a gas thereto; a reservoir coupled to the inlet and to the outlet; and a heat remover configured to cool a gas within the reservoir and to cause moisture within the gas to condensate within the reservoir.

### BRIEF DESCRIPTION OF THE DRAWINGS

FIG. 1 is a functional block diagram of one embodiment of a ventilation arrangement that includes a condenser in its exhalation path.

FIG. 2 shows a ventilator system for providing ventilation to a patient.

FIG. 3 is a functional block diagram of another embodiment of a ventilation arrangement that includes a condenser in its exhalation path.

FIGS. 4A-C illustrate various views of one embodiment of a condenser that may be employed in the exhalation path of a ventilator system.

FIG. 5 illustrates a cartridge assembly for a condenser.

### DETAILED DESCRIPTION

The present invention will now be described more fully hereinafter with reference to the accompanying drawings, in which preferred embodiments of the invention are shown. This invention may, however, be embodied in different forms and should not be construed as limited to the embodiments set forth herein. Rather, these embodiments are provided as teaching examples of the invention.

FIG. 1 is a functional block diagram of one embodiment of a ventilation arrangement 100 that includes a condenser in the exhalation path. Ventilation arrangement 100 includes a ventilator 110, an inhalation filter 120, a humidifier 130, a condenser 140, and an expiratory filter 150. The arrangement 100 may comprise a dual-limb patient circuit including an inhalation patient circuit or inspiratory limb, and an exhalation patient circuit or expiratory limb, both of which are connected to a patient.

FIG. 2 shows a ventilator system 200 for providing ventilation to a patient 10 using the elements of the ventilation arrangement 100. In particular, FIG. 2 better illustrates a dual-limb patient circuit 210 which includes an inspiratory limb 212, an expiratory limb 214, a Y-connector 217, and a breathing tube connected to inspiratory limb 212 and expiratory limb 214 via Y-connector 217. In some embodiments, the breathing tube may be an endotracheal tube. A pressure transducer 215 may be connected to Y-connector 217 for measuring a patient airway pressure provided to patient 10. FIG. 2 also shows tubing 205 connected between various elements in ventilator system 200. Tubing 205 may be conventional tubing employed in ventilator systems, for example tubing having an inner diameter of about 15 mm.

As a practical matter, in some installations ventilator 110 is provided as part of a facility's infrastructure (i.e., it may be installing in a hospital room), and the rest of ventilator system 200 may be separately provided for connection to ventilator 110.

Inhalation filter 120 filters a gas that is to be provided from ventilator 110 to patient 10. Humidifier 130 increases the humidity of the gas that is to be provided to patient 10 which may increase patient comfort. In some embodiments, humidifier 130 may be omitted. Expiratory filter 150 filters contaminants out of an exhaled gas that passes from patient 10 to ventilator 110, to reduce or eliminate any cross-contamination of ventilator 110 when it is used for different patients. Beneficially, expiratory filter 150 is a non-heated expiratory filter.

Beneficially, condenser 140 cools the exhaled gas from patient 10 and condenses some or substantially all of the moisture present in the exhaled gas so as to remove it from the exhaled gas, and passes the dried exhaled gas to expiratory filter 150. In ventilation arrangement 100, condenser 140 has one or more power and/or control inputs for receiving power and/or electrical control signals 105 from ventilator 110 which may be employed to adjust or control or more operating parameters of condenser 140 (e.g., to set a cooling temperature applied by condenser for removing moisture from the exhaled gas).

The inclusion of condenser 140 may provide one or more of the following beneficial features for ventilation arrangement 100: it may provide a dry gas to expiratory filter 150 so that expiratory filter 150 does not absorb moisture and require frequent replacement; it may prevent a large pressure drop across expiratory filter 150 in expiratory limb 214; it may permit the use of a simple and cheap anti-bacterial element for expiratory filter 150; it may consume less power than a heated expiratory filter. Also, in some embodiments as described below, condensed liquid may be removed from the patient circuit without breaking the patient circuit, as is required in existing ventilator systems that employ a non-heated expiratory filter. Furthermore, in some embodiments as will be described below, condenser 140 may be employed in ventilator systems which do not include any controls for a heated expiratory filter.

FIG. 3 is a functional block diagram of another embodiment of a ventilation arrangement 300 that includes a condenser 340 in its exhalation path.

Ventilation arrangement 300 is the same as ventilation 100, except for the following differences. Ventilation arrangement 300 includes a ventilator 310 that does not provide power and/or electrical control signals to condenser 340. Condenser 340 may be connected directly to an electrical outlet for receiving electrical power. Condenser 340 may also include a manual control 342 for receiving a manual control input from a user for adjusting one or more operating parameters of condenser 340 (e.g., a knob for allowing a user to set a cooling temperature applied by condenser 340 to the exhaled gas).

An advantage of ventilation arrangement 300 is that it may be configured to operate with an existing, installed, ventilator 310 which does not have any capability for controlling or adjusting any parameters of a condenser.

Other embodiments of a condenser may include both: (1) power and/or control input(s) for receiving power and/or electrical control signals 105 from a ventilator, and (2) an input for receiving power directly from an electrical outlet and a manual control for receiving a manual control input from a user for adjusting one or more operating parameters of the condenser. In this way, one condenser unit may be installed in either a ventilation arrangement where the ventilator can provide electrical controls, or a ventilation arrangement where the ventilator cannot provide electrical controls, and any operating parameters must be adjusted or set by a user.

FIGS. 4A-C illustrate various views of one embodiment of a condenser 400 that may be employed in the exhalation path of a ventilator system, such as ventilation system 200.

Condenser 400 includes a gas flow unit 410, a reservoir 420, a heat remover 430, and a thermally conductive cold plate 440.

Gas flow unit 410 has an inlet 412, an outlet 414, an exhaust tube 416, and a spiral fin 418 disposed on an outer surface of exhaust tube 416.

Reservoir 420 includes a canister 422, a disposable liner (e.g., a plastic bag) 424 removably disposed within canister 422, and an O-Ring 426 coupling disposable liner 424 to canister 422. Exhaust tube 416 and spiral fin 418 are disposed within disposable liner 424, canister 422 and reservoir 420.

In a beneficial embodiment, heat remover 430 comprises a thermoelectric cooling device, although it is conceivable that a different form of heat removing device may be employed.

Thermally conductive cold plate 440 may comprise a material with good thermal conductivity properties, such as aluminum. Thermally conductive cold plate 440 is thermally coupled to heat remover 430 and to canister 422. In a beneficial arrangement, thermally conductive cold plate 440 substantially surrounds reservoir 420, except for at the top opening of reservoir 420 into which gas flow unit 410 is disposed. In a beneficial arrangement, thermally conductive cold plate 440 has a cavity formed therein for receiving and substantially surrounding canister 422.

In some embodiments, canister 422 is removably disposed within the cavity of thermally conductive cold plate 440.

In some embodiments, gas flow unit 410, canister 422, disposable liner 424 and O-Ring 426 may together comprise a cartridge assembly that may be removed in one piece from thermally conductive cold plate 440, which functions as a housing for the cartridge assembly. FIG. 5 illustrates one embodiment of such a cartridge assembly 500.

In operation, inlet 412 receives exhaled gas from patient 10 via an expiratory limb of a patient circuit, and provides the exhaled gas to the interior of reservoir 420, and particularly to the interior of disposable liner 424. Heat remover 430 is configured, in conjunction with thermally conductive cold plate 440, to cool the exhaled gas within reservoir 420 (i.e., disposable liner 424). In some embodiments, one or more operations of heat remover 430 (e.g., an operating temperature) may be adjusted or controlled in response to one or more control signals or manual inputs provided to condenser 400, as described above with respect to FIGS. 1-3. When the exhaled gas is cooled by heat remover 430 and thermally conductive cold plate 440, moisture within the exhaled gas condenses into a liquid and is retained within reservoir 420 (i.e., within disposable liner 424). Beneficially, substantially all of the moisture with the exhaled gas is collected within reservoir 420 (i.e., within disposable liner 424). Then, the cooled and dried exhaled gas is output from condenser 430 via outlet 414, which may be connected to an exhalation filter in the expiratory limb of a patent circuit.

In a beneficial feature, one or more spiral fins 428 are disposed in a gas flow path between inlet 412 and outlet 414 such that at least a portion of the exhaled gas from patient 10 passes along the spiral fin in a path from inlet 412 to outlet 414. Spiral fins 428 cool the exhaled gas as it passes along spiral fins 428, thereby condensing moisture from the gas. Some or all of the moisture from the exhaled gas may, for example, form condensation on spiral fins 418, which in turn drops into disposable liner 424.

In another beneficial feature, canister 422 may include a monitor window 450 (e.g., glass or transparent plastic) through which an interior of canister 422 may be viewed from outside canister 422 when canister 422 is disposed within the cavity of thermally conductive cold plate 440. Further, disposable liner 424 may comprise a transparent or translucent material which permits its contents to be viewed from the outside. In this way, a user may be able to easily discern the amount of moisture which has accumulated within disposable liner 424, and thereby ascertain when disposable liner 424 should be replaced.

Beneficially, cartridge assembly 500 may be removed from condenser 400 and a used disposable liner may be exchanged for a new disposable liner 424 without breaking the patient circuit for patient 10.

Condenser 400 may provide one or more of the following beneficial features for a ventilator system in which it is employed: it may provide a dry gas to a subsequent expiratory filter so that the expiratory filter does not absorb moisture and require frequent replacement; it may prevent a large pressure drop across the expiratory filter which could otherwise require increased effort for a patient to breathe; it may permit the use of a simple and cheap anti-bacterial expiratory filter; it may consume less power than a heated expiratory filter; it may allow condensed liquid to be removed from the patient circuit without breaking the patient circuit; and it may be employed in ventilator systems which do not include any controls for a heated expiratory filter.

While preferred embodiments are disclosed herein, many variations are possible which remain within the concept and scope of the invention. Such variations would become clear to one of ordinary skill in the art after inspection of the specification, drawings and claims herein. The invention therefore is not to be restricted except within the scope of the appended claims.

1. A system, comprising:
an inhalation filter configured to receive and filter a gas;
a humidifier connected to the inhalation filter and configured to adjust a humidity of the filtered gas;
a dual-limb patient circuit connected to a patient and configured to supply ventilation to the patient, the dual limb patient circuit including an inspiratory limb connected to the humidifier and configured to supply the filtered gas to the patient, and further including an expiratory limb configured to receive an exhaled gas from the patient;
a condenser including a canister configured to receive a disposable liner removably disposed within the canister, wherein the canister is connected to the expiratory limb and configured to cool and remove moisture from the exhaled gas from the patient, further wherein moisture removed from the exhaled gas is retained within the disposable liner; and
an expiratory filter connected to the condenser and configured to filter the cooled exhaled gas from the condenser.

2. The system of claim 1, wherein the condenser comprises:
an inlet configured to receive the exhaled gas from the patient;
an outlet configured to output the cooled exhaled gas from the condenser;
a reservoir connected to the inlet and to the outlet; and
a heat remover configured to cool the exhaled gas within the reservoir.

3. The system of claim 2, wherein the heat remover comprises a thermoelectric cooling device.

4. The system of claim 3, further comprising a thermally conductive cold plate thermally coupled to the thermoelectric cooling device and substantially surrounding the reservoir.

5. The system of claim 4, wherein the reservoir comprises:
the canister disposed within a cavity formed by the thermally conductive cold plate;
the disposable liner removably disposed within the canister; and
an O-Ring coupling the disposable liner to the canister.

6. The system of claim 5, further comprising a spiral fin disposed within the disposable liner such that at least a portion of the exhaled gas from the patient passes along the spiral fin in a path from the inlet to the outlet.

7. The system of claim 6, further comprising a gas flow unit comprising the inlet, the outlet, the spiral fin, and an exhaust tube connected to the inlet and disposed within the disposable liner through which the exhaled gas from the patient passes into the disposable liner, and wherein the spiral fin is provided on an external surface of the exhaust tube.

8. The system of claim 3, wherein the condenser includes a control input configured to receive a control signal from a ventilator for controlling a cooling operation of the thermoelectric cooling device.

9. The system of claim 3, wherein the condenser includes a manual control for controlling a cooling operation of the thermoelectric cooling device, and wherein the manual control is configured to be manually adjusted by a user.

10. The system of claim 1, further comprising a ventilator connected to the inhalation filter and to the expiratory filter, and configured to provide the gas to the inhalation filter and to receive the filtered exhaled gas from the expiratory filter.

11. An apparatus, comprising:
an inlet configured to be connected to an expiratory limb of a dual-limb patient circuit;
an outlet configured to be connected to an expiratory filter of a ventilator system;
a thermoelectric cooling device;
a thermally conductive cold plate thermally coupled to the thermoelectric cooling device and having a cavity formed therein;
a reservoir disposed within the cavity and coupled to the inlet and to the outlet; and
a spiral fin disposed within the reservoir in a gas flow path from the inlet to the outlet.

12. The apparatus of claim 11, wherein the reservoir comprises:
a canister disposed within a cavity formed by the thermally conductive cold plate;
a disposable liner removably disposed within the canister; and
an O-Ring coupling the disposable liner to the canister.

13. The apparatus of claim 12, wherein the canister includes a monitor window through which an interior of the canister may be viewed from outside the canister when the canister is disposed within the cavity.

14. The apparatus of claim 11, further comprising a gas flow unit comprising the inlet, the outlet, the spiral fin, an exhaust tube connected to the inlet and disposed within the reservoir through which the exhaled gas from the patient passes into the reservoir, and wherein the spiral fin is provided on an external surface of the exhaust tube.

15. The apparatus of claim 11, further comprising a control input configured to receive a control signal for controlling a cooling operation of the thermoelectric cooling device.

16. The apparatus of claim 11, further comprising a manual control for controlling a cooling operation of the thermoelectric cooling device, and wherein the manual control is configured to be manually adjusted by a user.

17. An apparatus, comprising:
an inlet configured to be connected to an expiratory limb of a dual-limb patient circuit and configured to receive a gas therefrom;
an outlet configured to be connected to an expiratory filter of a ventilator system and configured to provide a gas thereto;
a reservoir including a canister configured to receive a disposable liner removably disposed within the canister, wherein the reservoir is coupled to the inlet and to the outlet; and
a heat remover configured to cool a gas within the reservoir and to cause moisture within the gas to condensate, wherein the moisture is retained within the disposable liner removably disposed with the canister of the reservoir.

18. The apparatus of claim 17, further comprising a spiral fin disposed within the reservoir in a gas flow path from the inlet to the outlet.

19. The apparatus of claim 18, wherein the reservoir comprises:
the canister;
the disposable liner removably disposed within the canister; and
an O-Ring coupling the disposable liner to the canister.

20. The apparatus of claim 19, further comprising a gas flow unit comprising the inlet, the outlet, the spiral fin, an exhaust tube connected to the inlet and disposed within the disposable liner through which the exhaled gas from the patient passes into the reservoir, wherein the spiral fin is provided on an external surface of the reservoir.
